(19) United States
(12) Patent Application Publication
(10) Pub. No.: US 2023/0044549 A1
(43) Pub. Date: Feb. 9, 2023

(54) COMPOSITION FOR CONDITIONING KERATAIN FIBERS

(71) Applicant: L'OREAL, Paris (FR)

(72) Inventors: Junliang ZHANG, Shanghai (CN); Yi HUANG, Shanghai (CN); Xiaoyin ZHOU, Shanghai (CN); Jessica YUAN, Shanghai (CN); Amit JAYASWAL, Shanghai (CN)

(73) Assignee: L'OREAL, Paris (FR)

(21) Appl. No.: 17/788,604

(22) PCT Filed: Dec. 25, 2019
(86) PCT No.: PCT/CN2019/128218

§ 371 Date: Jun. 23, 2022

(51) Int. Cl. A61K 8/892; A61K 8/37; A61K 8/33; A61Q 5/12

(52) U.S. Cl. CPC A61K 8/892; A61K 8/37; A61K 8/33; A61Q 5/12

(57) ABSTRACT

It relates to a composition for conditioning keratin fibers comprising in an oily phase: a) at least one silicone resin; b) at least one triglyceride; and c) at least one dialkyl ether. It also relates to a cosmetic process for caring for keratin fibers comprising at least a step of applying the composition onto said keratin fibers.

### TECHNICAL FIELD

The present invention relates to a composition for conditioning keratin fibers. The present invention also relates to a cosmetic process for conditioning keratin fibers.

### BACKGROUND ART

The appearance and/or condition of keratin substrates, for example, keratin fibers such as hair, skin, nails, and lips, are often affected by both extrinsic and intrinsic factors such as aging. In particular, when keratin substrates are exposed to environmental conditions, for example, high or low humidity or to ultraviolet radiation from the sun, these substrates can lose many of their desirable properties and even become damaged. Keratin fibers, especially hair, are constantly exposed to harsh extrinsic conditions, such as sun, chemical damage, e.g., from detergents, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. Furthermore, hair can lose its desirable attributes due to physical stress on the hair such as brushing and application of heat. As such, these factors generally result in damage to the keratin fibers, either by affecting protective materials on the surface of the hair (the cuticle), or by altering the hair fiber internally (the cortex).

Thus, it is highly desirable to have products providing the hair conditioning benefits.

It is common to resort to a hair conditioner or hair masque (mask) product, which can be used on hair after it has been shampooed or rinsed in order to confer hair caring properties to hair. Traditional conditioners and masques provide conditioning, smoothing, softening and visual sleekness to hair and are generally rinse-off products.

However, some ingredients can be easily removed from the hair, for example by rinsing or washing. Thus, any cosmetic benefits to the hair from such products are generally diminished or removed once the hair is rinsed or washed.

Thus, there is still a need to develop compositions for conditioning the hair, which would effectively provide the hair conditioning benefits and give the hair a light and clean feel.

### SUMMARY OF THE INVENTION

An object of the present invention is thus to develop a composition for conditioning the hair, which would effectively provide the hair conditioning benefits and give the hair a light and clean feel.

Thus, according to a first aspect, the present invention provides a composition for conditioning keratin fibers comprising in an oily phase:

a) at least one silicone resin;

b) at least one triglyceride; and

c) at least one dialkyl ether.

According to a second aspect, the present invention provides a cosmetic process for conditioning keratin fibers comprising applying the composition as described above onto the keratin fibers.

The inventors have found that with the combination of a) at least one silicone resin, b) at least one triglyceride, and c) at least one dialkyl ether, the composition would effectively provide keratin fibers conditioning benefits and give the keratin fibers a light and clean feel.

Other characteristics and advantages of the invention will emerge more clearly on reading the description and the examples that follow.

### DETAILED DESCRIPTION OF THE INVENTION

As used herein, unless otherwise indicated, the limits of a range of values are included within this range, in particular in the expressions “between . . . and . . . ” and “from . . . to . . . ”.

As used herein, the term “comprising” is to be interpreted as encompassing all specifically mentioned features as well optional, additional, unspecified ones.

As used herein, the use of the term “comprising” also discloses the embodiment wherein no features other than the specifically mentioned features are present (i.e. “consisting of”).

Unless otherwise defined, all technical and scientific terms used herein have the same meaning as commonly understood by those skilled in the field the present invention belongs to. When the definition of a term in the present description conflicts with the meaning as commonly understood by those skilled in the field the present invention belongs to, the definition described herein shall apply.

Unless otherwise specified, all numerical values expressing amount of ingredients and the like used in the description and claims are to be understood as being modified by the term “about”. Accordingly, unless indicated to the contrary, the numerical values and parameters described herein are approximate values which are capable of being changed according to the desired performance obtained as required.

As used herein, the expression “at least one” used in the present description is equivalent to the expression “one or more” and may be substituted for it.

As used herein, the term “keratin fibers” includes animal keratin fibers and human keratin fibers such as the hair.

According to the first aspect of the present invention, a composition for conditioning keratin fibers comprises in an oily phase:

a) at least one silicone resin;

b) at least one triglyceride; and

c) at least one dialkyl ether.

Silicone Resin

According to the first aspect, the composition according to the invention comprises at least one silicone resin.

More generally, the term “resin” means a compound whose structure is three-dimensional. Thus for the purposes of the present invention, a polydimethylsiloxane is not a silicone resin.

The nomenclature of silicone resins (also known as siloxane resins) is known under the name “MDTQ”, the resin being described as a function of the various siloxane monomer units it comprises, each of the letters “MDTQ” characterizing a type of unit.

The letter M represents the monofunctional unit of formula R1R2R3SiO_{1/2}, the silicon atom being bonded to only one oxygen atom in the polymer comprising this unit.

The letter D means a difunctional unit R1R2SiO_{2/2} in which the silicon atom is bonded to two oxygen atoms.

The letter T represents a trifunctional unit of formula R1SiO_{3/2}.

Such resins are described, for example, in the Encyclopedia of Polymer Science and Engineering, vol. 15, John Wiley and Sons, New York, (1989), pp. 265-270, and U.S. Pat. Nos. 2,676,182, 3,627,851, 3,772,247, 5,248,739 or 5,082,706, 5,319,040, 5,302,685 and 4,935,484.

In the units M, D and T defined previously, Ri, namely R1, R2 and R3, which may be identical or different, represent a hydrocarbon-based radical (especially alkyl) containing from 1 to 10 carbon atoms, a phenyl group, a phenylalkyl group or else a hydroxyl group.

Finally, the letter Q means a tetrafunctional unit SiO_{4/2} in which the silicon atom is bonded to four oxygen atoms, which are themselves bonded to the rest of the polymer.

Various silicone resins with different properties may be obtained from these different units, the properties of these polymers varying as a function of the type of monomer (or unit), the nature and number of the radical(s) Ri, the length of the polymer chain, the degree of branching and the size of the side chains.

As silicone resins that may be used in the compositions according to the invention, use may be made, for example, of silicone resins of MQ type, of T type or of MQT type.

MQ Resins:

As examples of silicone resins of MQ type, mention may be made of the alkyl siloxysilicates of formula [(R1)₃SiO_{1/2}]ₓ(SiO_{4/2})_{y} (units MQ) in which x and y are integers ranging from 50 to 80, and such that the group R1 represents a radical as defined previously, and is preferably an alkyl group containing from 1 to 8 carbon atoms or a hydroxyl group, preferably a methyl group.

As examples of solid silicone resins of MQ type of trimethyl siloxysilicate type, mention may be made of those sold under the reference SR1000 by the company Momentive Performance Materials, under the reference MQ 1600 by Dow Corning or under the reference Belsil TMS 803 by the company Wacker.

As silicone resins comprising MQ siloxysilicate units, mention may also be made of phenylalkyl siloxysilicate resins, such as phenylpropyldimethyl siloxysilicate (Silshine 151 sold by the company Momentive Performance Materials). The preparation of such resins is described especially in U.S. Pat. No. 5,817,302.

T Resins:

Examples of silicone resins of T type that may be mentioned include the polysilsesquioxanes of formula (RSiO_{3/2})ₓ (units T) in which x is greater than 100 and such that the group R is an alkyl group containing from 1 to 10 carbon atoms, said polysilsesquioxanes also possibly comprising Si—OH end groups.

Mention may also be made of polymethylsilsesquioxanes, which are polysilsesquioxanes in which none of the methyl radicals is substituted with another group. Such polymethylsilsesquioxanes are described, for example, in U.S. Pat. No. 5,246,694.

Polymethylsilsesquioxane resins that may preferably be used are those in which R represents a methyl group, for instance those sold:
by the company Wacker under the reference Resin MK, such as Belsil PMS MK: polymer comprising CO₃SiO_{3/2} repeating units (units T), which may also comprise up to 1% by weight of (CH₃)₂SiO_{2/2} units (units D) and having an average molecular weight of about 10 000 g/mol, or
by the company Shin-Etsu under the references KR-220L, which are composed of units T of formula CH₃SiO_{3/2} and contain Si—OH (silanol) end groups, under the reference KR-242A, which comprise 98% of units T and 2%> of dimethyl units D and contain Si—OH end groups, or else under the reference KR-251, comprising 88% of units T and 12%> of dimethyl units D and contain Si—OH end groups.
by the company Dow Corning under the references Dow Corning 670 Fluid, Dow Corning 680 Fluid, as a mixture in cyclopentasiloxane and in isododecane, respectively.

MQT Resins:

Resins comprising MQT units that are especially known are those mentioned in U.S. Pat. No. 5,110,890.

A preferred form of resins of MQT type are MQT-propyl (also known as MQTpr) resins. Such resins that may be used in the compositions according to the invention are especially the resins described and prepared in patent application WO 2005/075 542, the content of which is incorporated herein by reference.

The MQ-T-propyl resin preferably comprises the following units:

(i) (R1₃SiO_{1/2})a

(ii) (R2₂SiO_{2/2})b

(iii) (R₃SiO_{3/2})c and

(iv) (SiO_{4/2})d

with
R1, R2 and R3 independently representing a hydrocarbon-based radical (especially alkyl) containing from 1 to 10 carbon atoms, a phenyl group, a phenylalkyl group or a hydroxyl group and preferably an alkyl radical containing from 1 to 8 carbon atoms or a phenyl group,
a, b, c and d being mole fractions,
a being between 0.05 and 0.5,
b being between zero and 0.3,
c being greater than zero,
d being between 0.05 and 0.6,
a+b+c+d=1,
on condition that more than 40 mol %> of the groups R³ of the siloxane resin are propyl groups.

Preferably, the siloxane resin comprises the following units:

(i) (R1₃SiO_{1/2})a

(iii) (R₃SiO_{3/2})c and

(iv) (SiO_{4/2})d

with
R1 and R3 independently representing an alkyl group containing from 1 to 8 carbon atoms, R1 preferably being a methyl group and R3 preferably being a propyl group,
a being between 0.05 and 0.5 and preferably between 0.15 and 0.4,
c being greater than zero, preferably between 0.15 and 0.4,
d being between 0.05 and 0.6, preferably between 0.2 and 0.6 or alternatively between 0.2 and 0.55,

a+b+c+d=1, and a, b, c and d being mole fractions,
on condition that more than 40 mol % of the groups R³ of the siloxane resin are propyl groups.

The siloxane resins that may be used according to the invention may be obtained via a process comprising the reaction of:

A) an MQ resin comprising at least 80 mol % of units (R1₃SiO_{1/2}) and (SiO_{4/2})d,
R1 representing an alkyl group containing from 1 to 8 carbon atoms, an aryl group, a carbinol group or an amino group,
a and d being greater than zero,
the ratio a/d being between 0.5 and 1.5,

and

B) a T-propyl resin comprising at least 80 mol % of units (R3SiO_{3/2})_{c},
R3 representing an alkyl group containing from 1 to 8 carbon atoms, an aryl group, a carbinol group or an amino group,
c being greater than zero,
on condition that at least 40 mol % of the groups R3 are propyl groups,
in which the mass ratio A/B is between 95/5 and 15/85 and preferably the mass ratio A/B is 30/70.

Advantageously, the mass ratio A/B is between 95/5 and 15/85. Preferably, the ratio A/B is less than or equal to 70/30. These preferred ratios have proven to afford comfortable deposits. Preferably, the composition according to the invention comprises, as silicone resin, at least one resin of MQ type as described previously.

In particular, the silicone resin is a siloxysilicate resin, preferably a trimethylsiloxysilicate resin.

The silicone resin may be used in powder form, in a form dissolved in a solvent, in a form conveyed in a liquid or in a form emulsified in water. It should be noted that, in the latter case, the silicone resin is preferably in a conveyed form, advantageously dissolved in a solvent, and then emulsified. Preferably the silicone resin is used in a form conveyed in a solvent, or in a form emulsified in water.

As regards the silicone resins conveyed in a solvent, said solvent is usually selected from volatile or non-volatile, apolar hydrocarbon-based oils and silicone oils, preferably volatileoils.

Volatile hydrocarbon-based oils that may especially be mentioned include alkanes, preferably branched alkanes of 8 to 16 carbon atoms, especially such as C8-C16 isoalkanes (also known as isoparaffins), isododecane, isodecane and isohexadecane.

Volatile silicone oils that may be mentioned include linear or cyclic silicone oils, such as linear or cyclic polydimethylsiloxanes (PDMSs) containing from 3 to 7 silicon atoms.

Examples of such oils that may be mentioned include octyl trimethicone, hexyl trimethicone, decamethylcyclopentasiloxane, octamethylcyclotetrasiloxane, dodecamethylcyclohexasiloxane, decamethyltetrasiloxane, methyl trimethicone, polydimethylsiloxanes such as those sold under the reference DC 200 by Dow Corning or KF 96 A from Shin-Etsu; alone or as mixtures.

Among the silicone resins, especially of MQ type, which are in a form conveyed in a solvent, mention may be made especially of Koboguard® MQ65TMF (mixture of tnmethylsiloxysilicate and methyl trimethicone) sold by Kobo; KF-7312J (mixture in cyclopentasiloxane), KF-7312K, KF-7312L (mixtures in dimethicone), KF-7312T (mixture in trimethicone), X-21-5249 (mixture in cyclopentasiloxane), X-21-5249L (mixture in dimethicone), X-21-5250, X-21-5250L (mixture in cyclopentasiloxane and dimethicone, respectively), X-21-5595, X-21-5616 (mixtures in isododecane), KF-9021, KF-9021L (mixtures in cyclopentasiloxane and in dimethicone, respectively), sold by Shin-Etsu; Silsoft 74, Silshine 151 (mixtures in isododecane) from Momentive Performance Materials; Xiameter RSN-0749 Resin, Dow Corning 749 Fluid (mixtures in cyclopentasiloxane), Dow Corning 593 Fluid (mixture in dimethicone) from Dow Corning.

As regards the silicone resins that are in the form of emulsions in water, mention may be made, for example, of KM-9717 (emulsion in the presence of an anionic surfactant, comprising a low-viscosity silicone), X-52-8005 (emulsion in the presence of a nonionic surfactant, comprising a low-viscosity silicone), sold by Shin-Etsu.

In a preferred embodiment, the silicone resin is selected from MQ type silicone resin.

If presents, the silicone resin is present in an amount ranging from 0.5 wt. % to 10 wt. %, preferably from 0.8 wt. % to 5 wt. %, more preferably from 1 wt. % to 4 wt. %, relative to the weight of the composition.

Triglyceride

According to the first aspect, the composition according to the invention comprises at least one triglyceride.

Preferably, the triglyceride has the following formula (I):

CH₂(OOCR₁)CH(OOCR₂)CH₂(OOCR₃) (I)

wherein R₁, R₂ and R₃ are independently chosen from C₆-C₃₀ alkyl and C₆-C₃₀ alkenyl.

Preferably, in formula (I), R₁, R₂ and R₃ are independently chosen from C₆-C₂₄ alkyl and C₆-C₂₄ alkenyl, preferably C₆-C₂₀ alkyl and C₆-C₂₀ alkenyl, more preferably C₆-C₁₄ alkyl and C₆-C₁₄ alkenyl, more preferably C₆-C₁₂ alkyl and C₆-C₁₂ alkenyl, most preferably C₆-C₁₀ alkyl and C₆-C₁₀ alkenyl, said alkyl or alkenyl is linear or branched.

In formula (I), R₁, R₂ and R₃ may be different, or two or all of R₁, R₂ and R₃ may be the same.

Examples of triglyceride according to formula (I) are given in the CTFA Cosmetic Ingredient Handbook.

Preferred triglyceride according to formula (I) are obtained from carboxylic acids of carbon chain length ranging from C₆ to C₂₄, preferably from C₆ to C₂₀, and more preferably from C₆ to C₁₈, linear or branched, saturated or unsaturated, and glycerol.

More preferably, the triglyceride according to formula (I) are chosen from triglycerides of fatty acids containing from 6 to 14 carbon atoms, more preferably from 6 to 12 carbon atoms, in particular from 6 to 10 carbon atoms such as triglycerides of heptanoic acid, 2-ethylhexanoic acid, octanoic acids, caprylic acid, capric acid, or mixtures thereof.

In one embodiment, the triglyceride according to formula (I) are synthetic.

In another embodiment, the triglyceride according to formula (I) are of plant origin. For example, the plant oils that comprise triglyceride according to formula (I), or triglyceride according to formula (I) obtained from the plant oils can be used.

Vegetable derived triglyceride according to formula (I) are particularly preferred, and specific examples of preferred materials as sources of triglyceride according to formula (I) include peanut oil, sesame oil, avocado oil, coconut oil, cocoa butter oil, almond oil, safflower oil, corn oil, cotton seed oil, olive oil, jojoba oil, palm oil, soybean oil, wheat germ oil, linseed oil, and sunflower seed oil.

Mentions maybe made of the canola oil, such as that sold under the tradename Lipex Preact by the company AARHUSKARL SHAMN.

Preferably, mentions can be made of caprylic/capric acid triglycerides, such as those sold by the company Stearineries Dubois or those sold under the names Miglyol® 810, 812 and 818, jojoba oil, and shea butter oil. Mentions may also be made of the product sold by the company Wilmar under the name Wilfare Ster MCT, with INCI name caprylic/capric triglyceride.

Advantageously, the triglyceride is present in the composition of the present invention in an amount ranging from 10 wt. % to 40 wt. %, more preferably from 15 wt. % to 30 wt. %, relative to the total weight of the composition.

Dialkyl Ether

According to the first aspect, the composition according to the invention comprises at least one dialkyl ether.

Preferably, the dialkyl ether has the following formula (II):

R₄—O—R₅ (II)

wherein:

R₄ and R₅, which may be identical or different, denote a linear or branched C₆-C₂₅ alkyl or alkenyl radical.

Preferably, the ether of formula (II) is chosen from compounds for which the radicals R₄ and R₅, which may be identical or different, denote a linear or branched C₆-C₁₂ alkyl or alkenyl radical.

More particularly, according to the present invention, the radicals R₄ and R₅ are identical.

In accordance with one particular embodiment of the invention, the preferred dialkyl ether is chosen from di-n-hexyl ether, di-n-heptyl ether, di-n-octyl ether, di-n-nonyl ether, di-n-decyl ether, di-isodecyl ether, di-n-dodecyl ether, di-n-teteradecyl ether, di-n-hexadecyl ether, di-n-oxtadecyl ether, or a mixture thereof.

R₄ and R₅ preferentially denote a C₈ radical.

These compounds may be prepared according to the process described in patent application DE 41 27 230.

Most preferably, the ether of formula (II) is di-n-octyl ether (INCI name: dicaprylyl ether). Such product is commercially available, for example those sold under the name Cetiol OE by the company Cognis (BASF), or Rofetan OE by the company Ecogreen Oleochemicals.

Advantageously, the dialkyl ether is present in the composition of the present invention in an amount ranging from 5 wt. % to 30 wt. %, more preferably from 8 wt. % to 18 wt. %, relative to the total weight of the composition.

Oily Phase

According to the first aspect of the present invention, the composition comprises an oily phase.

The oily phase may comprise additional oil different from the triglyceride and the dialkyl ether mentioned above.

Here, “oil” means a fatty compound or substance which is in the form of a liquid or a paste (non-solid) at room temperature (25° C.) under atmospheric pressure (760 mmHg). As the oil(s), those generally used in cosmetics can be used alone or in combination thereof. These oil(s) may be volatile or non-volatile, preferably non-volatile.

The oil may be a hydrocarbon oil, a silicone oil, or the like.

As examples of hydrocarbon oils, mention may be made of alkane oils such as isododecane and isohexadecane, and ester oils different from the triglyceride mentioned above.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be selected especially from the group comprising the esters or mixtures of esters of sugars described previously and of linear or branched, saturated or unsaturated C₆-C₃₀ and preferably C₁₂-C₂₂ fatty acids.

If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, silicone oil is selected from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly selected from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

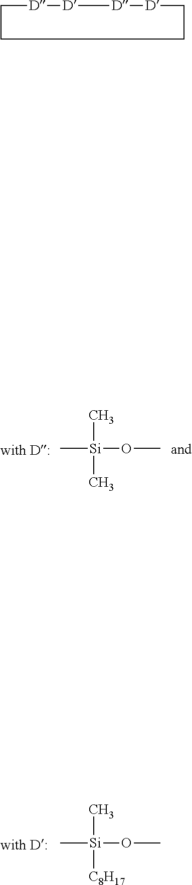

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly selected from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be selected from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

The term “fatty” in the fatty alcohol means the inclusion of a relatively large number of carbon atoms. Thus, alcohols which have 4 or more, preferably 6 or more, and more preferably 12 or more carbon atoms are encompassed within the scope of fatty alcohols. The fatty alcohol may be saturated or unsaturated. The fatty alcohol may be linear or branched.

The fatty alcohol may have the structure R—OH wherein R is selected from saturated and unsaturated, linear and branched radicals containing from 4 to 40 carbon atoms, preferably from 6 to 30 carbon atoms, and more preferably from 12 to 20 carbon atoms. In at least one embodiment, R may be selected from C₁₂-C₂₀ alkyl and C₁₂-C₂₀ alkenyl groups. R may be or may not be substituted with at least one hydroxyl group.

As examples of the fatty alcohol, mention may be made of lauryl alcohol, cetyl alcohol, stearyl alcohol, isostearyl alcohol, behenyl alcohol, undecylenyl alcohol, myristyl alcohol, octyldodecanol, hexyldecanol, oleyl alcohol, linoleyl alcohol, palmitoleyl alcohol, arachidonyl alcohol, erucyl alcohol, and mixtures thereof.

It is preferable that fatty alcohol be a saturated fatty alcohol.

Thus, the fatty alcohol may be selected from straight or branched, saturated or unsaturated C₆-C₃₀ alcohols, preferably straight or branched, saturated C₆-C₃₀ alcohols, and more preferably straight or branched, saturated C₁₂-C₂₀ alcohols.

The term “saturated fatty alcohol” here means an alcohol having a long aliphatic saturated carbon chain. It is preferable that the saturated fatty alcohol be selected from any linear or branched, saturated C₆-C₃₀ fatty alcohols. Among the linear or branched, saturated C₆-C₃₀ fatty alcohols, linear or branched, saturated C₁₂-C₂₀ fatty alcohols may preferably be used. Any linear or branched, saturated C₁₆-C₂₀ fatty alcohols may be more preferably used. Branched C₁₆-C₂₀ fatty alcohols may be even more preferably used.

As examples of saturated fatty alcohols, mention may be made of isostearyl alcohol, octyldodecanol, hexyldecanol, and mixtures thereof. In one embodiment, octyldodecanol, hexyldecanol, or a mixture thereof, can be used as a saturated fatty alcohol.

According to at least one embodiment, the fatty alcohol used in the composition according to the present invention is preferably selected from octyldodecanol, hexyldecanol and mixtures thereof.

It is preferable that the additional oil be selected from alkane oils, silicone oils, and a mixture thereof.

In a preferred embodiment, the addition oil is selected from branched alkane oils containing from 8 to 16 carbon atoms and better still from 10 to 16 carbon atoms such as isododecane, polydimethylsiloxanes optionally containing dimethylsilanol end groups, such as dimethicones, dimethiconols, and a mixture thereof.

In a preferred embodiment, the composition according to the present invention comprises from 50 wt. % to 60 wt. % of branched alkane oils containing from 8 to 16 carbon atoms.

In a preferred embodiment, the composition according to the present invention comprises from 50 wt. % to 60 wt. % of branched alkane oils containing from 8 to 16 carbon atoms and from 10 wt. % to 20 wt. % of polydimethylsiloxanes optionally containing dimethylsilanol end groups.

Advantageously, the total amount of the oil including the triglyceride and the dialkyl ether mentioned above ranges from 90 wt. % to 99 wt. %, preferably from 96 wt. % to 98 wt. %, relative to the total weight of the composition.

It is preferable that the composition according to the present invention be anhydrous.

The term “anhydrous” here means that the composition according to the present invention may contain only a small amount of water, preferably no water. Thus, the amount of water may be 2 wt. % or less, preferably 1.5 wt. % or less, and more preferably 1 wt. % or less relative to the total weight of the composition. It is particularly preferable that the cosmetic composition according to the present invention contains no water.

Additional Ingredients

The composition according to the present invention is advantageously a cosmetic composition.

The composition according to the present invention may also comprise an effective amount of other ingredients, known previously elsewhere in cosmetic compositions, such as various common adjuvants, vitamins or provitamins, for instance, panthenol, opacifiers, fragrances, plant extracts, thickeners, cationic polymers and so on.

According to a preferred embodiment, the present invention provides a composition for conditioning keratin fibers comprising, relative to the total weight of the composition:

a) from 1 wt. % to 4 wt. % of at least one MQ type silicone resin;

b) from 15 wt. % to 30 wt. % of at least one triglyceride selected from those having the following formula (I):

CH₂(OOCR₁)CH(OOCR₂)CH₂(OOCR₃) (I)

wherein R₁, R₂ and R₃ are independently chosen from C₆-C₁₀ alkyl and C₆-C₁₀ alkenyl;

c) from 8 wt. % to 18 wt. % of at least one dialkyl ether selected from those having the following formula (II):

R₄—O—R₅ (II)

wherein:

R₄ and R₅, which may be identical or different, denote linear or branched C₆-C₁₂ alkyl or alkenyl radical; and

d) 50 wt. % to 60 wt. % of at least one branched alkane oils containing from 8 to 16 carbon atoms.

Preparation and Use

The composition according to the present invention can be prepared by mixing ingredients a) to d), as essential ingredients, as well as optional ingredient(s), as explained above.

The method and means to mix the above essential and optional ingredients are not limited. Any conventional method and means can be used to mix the above essential and optional ingredients to prepare the composition according to the present invention.

The composition according to the present invention can be uniform.

The composition according to the present invention can be a conditioner, a leave-on product, for example, a leave-on oil.

The use of the composition can be done on wet or dry hair.

According to the second aspect of the present invention, a cosmetic process for conditioning keratin fibers comprising applying the composition as described above onto the keratin materials.

The following examples are given by way of illustration of the present invention and shall not be interpreted as limiting the scope.

### EXAMPLES

### Example 1: Formulation of Leave-on Oils

Leave-on oils according to invention formulas (Inv.) 1 and comparative formulas (comp.) 1-3 were prepared (the contents are expressed as weight percentages of active material with regard to the total weight of each leave-on oil, unless otherwise indicated):

| INCI US | Inv. 1 | Comp. 1 | Comp. 2 | Comp. 3 |
|---|---|---|---|---|
| ISODODECANE(Isododecane from INEOS) | QS 100 | QS 100 | QS 100 | QS 100 |
| CAPRYLIC/CAPRIC TRIGLYCERIDE(DUB MCT | 20 | 20 | 20 | 20 |
| 7030/MB from STEARINERIE DUBOIS) | | | | |
| DICAPRYLYL ETHER(CETIOL ® OE | 11 | — | — | 11 |
| from BASF) | | | | |
| C12-15 ALKYL BENZOATE(BENZOATE | — | 11 | — | — |
| DE C12/C15 (DUB B1215) from | | | | |
| STEARINERIE DUBOIS) | | | | |
| DIMETHICONE 5 CST(BELSIL ® DM 5 PLUS | 4 | 4 | 4 | 4 |
| DIMETHICONE from WACKER) | | | | |
| DIMETHICONE 10 CST (BELSIL DM 10 | — | — | 8 | — |
| DIMETHICONE from WACKER) | | | | |
| DIMETHICONE 500 000 CST (XIAMETER ® | 4 | 4 | 4 | 4 |
| PMX-200 SILICONE FLUID 500000 CST | | | | |
| from DOW CORNING (DOW CHEMICAL)) | | | | |
| TRIMETHYLSILOXYSILICATE(DOWSIL ™ | 2 | 2 | 2 | — |
| 593 FLUID from DOW CORNING (DOW | | | | |
| CHEMICAL)) | | | | |
| DIMETHICONOL(DOWSIL ™ XX-1515 GUM | 6 | 6 | 6 | 6 |
| from DOW CORNING (DOW CHEMICAL)) | | | | |

Leave-on oil of comparative formula 1 does not comprise any dialkyl ether.

Leave-on oil of comparative formula 2 does not comprise any dialkyl ether.

Leave-on oil of comparative formula 3 does not comprise any silicone resin.

The leave-on oils listed above were prepared by cold mixing all ingredients together evenly.

### Example 2: Evaluation of Leave-on Oils

The conditioning effect, light and clean feel of the hair treated with leave-on oils prepared in Example 1 were evaluated and scored as follows.

6 volunteers were recruited with highly damaged hair (hair treated with color, straightening/color, or perm wave for more than 3 times in the past 12 months). 10-12 g of shampoo was applied by sensorial experts on the whole head, after which the hair was separated into 2 parts with the leave-on oil to be tested and a commercialized leave-on oil (Loréal Extraordinary Oil) separately applied by sensorial experts on each part. When the procedure ended, sensorial experts evaluated hair status and gave scores accordingly.

5—overperformance difference is at least great enough to be noticeable by trained experts after 1-2 touch, as compared with the commercialized leave-on oil;

4—overperformance determined by direct comparison after 3 or more touch of trained expert, as compared with the commercialized leave-on oil;

3—similar performance with the commercialized leave-on oil;

2—underperformance determined by direct comparison after 3 or more touch of trained expert, as compared with the commercialized leave-on oil;

1—underperformance difference is at least great enough to be noticeable by trained experts after 1-2 touch, as compared with the commercialized leave-on oil.

Conditioning Benefits

The conditioning benefits were evaluated in terms of the regularity degree of the hair and easiness of fingers sliding through hair without hindrance perception and resistance.

Light and Clean Feel

The intensity of deposit and residue on hair were observed. More deposit and residue on hair indicates less lightness and cleanness.

The results for each composition were summarized in the following table.

| | Inv. 1 | Comp. 1 | Comp. 2 | Comp. 3 |
|---|---|---|---|---|
| Conditioning | 5 | 3 | 3 | 2 |
| Light and clean feel | 4 | 2 | 2 | 3 |

It can be seen from above table that the leave-on oil according to the present invention (Inv. 1) can effectively provide the hair conditioning benefits and give the hair a light and clean feel.

1: A composition for conditioning keratin fibers comprising in an oily phase:
a) at least one silicone resin;
b) at least one triglyceride; and
c) at least one dialkyl ether.

2: The composition according to claim 1, wherein the composition is anhydrous.

3: The composition according to claim 1, wherein the silicone resin is a MQ type silicone resin.

4: The composition according to claim 1, wherein the silicone resin is a siloxysilicate resin.

5: The composition according to claim 1, wherein the silicone resin is present in an amount ranging from 0.5 wt. % to 10 wt. % relative to the weight of the composition.

6: The composition according to claim 1, wherein the triglyceride is of formula (I)
CH2(OOCR1)CH(OOCR2)CH2(OOCR3) (I)
wherein R1, R2 and R3 are each independently selected from C6-C30 alkyl and C6-C30 alkenyl.

7: The composition according to claim 1, wherein the triglyceride is present in an amount ranging from 10 wt. % to 40 wt. % relative to the total weight of the composition.

8: The composition according to claim 1, wherein the dialkyl ether is of formula (II):
R₄—O—R₅ (II)
wherein:
R₄ and R₅, which may be identical or different, denote a linear or branched C6-C25 alkyl or alkenyl radical.

9: The composition according to claim 1, wherein the dialkyl ether is present in an amount ranging from 5 wt. % to 30 wt. % relative to the total weight of the composition.

10: The composition according to claim 1, further comprising an oil which is selected from branched alkane oils containing from 8 to 16 carbon atoms, polydimethylsiloxanes optionally containing dimethylsilanol end groups, and a mixture thereof.

11: The composition according to claim 1, further comprising from 50 wt. % to 60 wt. % of branched alkane oils containing from 8 to 16 carbon atoms and from 10 wt. % to 20 wt. % of polydimethylsiloxanes optionally containing dimethylsilanol end groups.

12: The composition according to claim 1, which is a leave-on oil.

13: A composition for conditioning keratin fibers comprising, relative to the total weight of the composition:
a) from 1 wt. % to 4 wt. % of at least one MQ type silicone resin;
b) from 15 wt. % to 30 wt. % of at least one triglyceride of formula (I):
CH2(OOCR1)CH(OOCR2)CH2(OOCR3) (I)
wherein R1, R2 and R3 are each independently selected from C6-C10 alkyl and C6-C10 alkenyl;
c) from 8 wt. % to 18 wt. % of at least one dialkyl ether of formula (II):
R₄—O—R₅ (II)
wherein:
R4 and R5, which may be identical or different, denote linear or branched C6-C12 alkyl or alkenyl radical; and
d) 50 wt. % to 60 wt. % of at least one branched alkane oil oils containing from 8 to 16 carbon atoms.

14: A cosmetic process for conditioning keratin fibers comprising applying a composition as defined in claim 1 onto said keratin fibers.
